(19) United States
(12) Patent Application Publication
(10) Pub. No.: US 2026/0115483 A1
(43) Pub. Date: Apr. 30, 2026

(54) DETACHABLE STENT-LEAD COMBINATION

(71) Applicant: Covidien LP, Mansfield, MA (US)

(72) Inventors: Robert T. Sandgren, Lindstrom, MN (US); Thaddeus S. Brink, St Paul, MN (US); Robert L. Olson, White Bear Lake, MN (US); Dale F. Seeley, Spring Park, MN (US)

(21) Appl. No.: 19/367,493

(22) Filed: Oct. 23, 2025

Related U.S. Application Data
(60) Provisional application No. 63/711,447, filed on Oct. 24, 2024.

(51) Int. Cl. A61N 1/375; A61N 1/05

(52) U.S. Cl. CPC A61N 1/37516; A61N 1/05; A61N 1/37518

(57) ABSTRACT

Systems, devices, and methods for electrical stimulation and removal of electrical stimulation components are disclosed herein. According to some embodiments, the present technology includes a medical device assembly including an expandable member configured to be positioned in a blood vessel. The medical device assembly can further include a lead body having a plurality of electrodes in a distal end region of the lead body, where the electrodes are configured to deliver therapeutic stimulation to surrounding tissue. The medical device assembly can further include one or more engagement members configured to releasably couple the lead body to the expandable member.

### CROSS REFERENCE TO RELATED APPLICATIONS

The present application claims priority to U.S. Provisional Patent Application No. 63/711,447, filed October 24, 2024, which is incorporated by reference herein in its entirety.

### TECHNICAL FIELD

The present technology generally relates to medical devices such as detachable stent-lead combinations for delivering stimulation from within the vasculature.

### BACKGROUND

Electrical stimulation can be an effective therapy for a variety of indications. Current stimulation therapies, such as those targeting neural structures, utilize semi-permanent or permanent medical devices (e.g., implanted leads). These devices are costly and require invasive procedures for implantation and maintenance. The vasculature may provide a broad network of accessible stimulation targets using minimally-invasive procedures. However, current vascular approaches risk thrombosis, vessel occlusion, and/or device failure due to the intransient placement of electrical hardware within the vasculature. For instance, some therapies may span multiple weeks, causing stimulation devices to remain in the body even when stimulation is not active. Further, current approaches may lack sufficient precision and stability within intricate vascular networks. Accordingly, there remains a need for minimally-invasive electrical stimulation devices with improved ease of removal, safety, and precision.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic illustration of an example treatment system, in accordance with embodiments of the present technology.

FIGS. 1B and 1C are block diagrams of example signal generators, in accordance with embodiments of the present technology.

FIGS. 2A and 2B are schematic side views of a medical device assembly deployed at a treatment site, in accordance with embodiments of the present technology.

FIGS. 3A-3E are schematic side views of a medical device assembly in various configurations, in accordance with embodiments of the present technology.

FIGS. 4A-4E are schematic side views of a medical device assembly in various configurations, in accordance with embodiments of the present technology.

FIG. 5 is a schematic side view of a medical device assembly, in accordance with embodiments of the present technology.

FIGS. 6A and 6B are schematic side and end views, respectively, of a medical device assembly, in accordance with embodiments of the present technology.

FIGS. 7A and 7B are schematic side and end views, respectively, of a medical device assembly, in accordance with embodiments of the present technology.

FIG. 8 is a schematic side view of a medical device assembly, in accordance with embodiments of the present technology.

FIG. 9 is a schematic side view of a medical device assembly, in accordance with embodiments of the present technology.

FIGS. 10A and 10B are schematic side views of medical device assemblies, in accordance with embodiments of the present technology.

FIG. 11 is a flowchart of an example method of using a medical device assembly for delivering electrical stimulation, in accordance with variations of the present technology.

### DETAILED DESCRIPTION

The present technology provides systems, devices, and methods for electrical stimulation of tissues from within the vasculature and safe and efficient removal of electrical components after therapy. Although many of the embodiments are described below with respect to systems, devices, and methods for delivering electrical signals to a cerebral or intracranial (e.g., neural) target, other applications and other embodiments in addition to those described herein are within the scope of the technology. For example, the treatment systems and methods of the present technology may be used to stimulate tissue from body lumens other than blood vessels (e.g., the digestive tract, etc.) and/or may be used to stimulate tissue from blood vessels outside of the brain (e.g., cardiac, pulmonary, abdominal, cervical, or thoracic blood vessels, or peripheral blood vessels including those within the legs or arms, etc.).

In some embodiments, for example, the present technology includes a medical device assembly including an expandable member (e.g., a stent) configured to be positioned in a blood vessel at a treatment site. The medical device assembly can further include a lead body having a plurality of electrodes in a distal end region of the lead body, where the electrodes are configured to deliver therapeutic stimulation to surrounding tissue. The medical device assembly can also include one or more engagement members configured to releasably couple the lead body to the expandable member. For instance, the one or more engagement members can include a receptacle positioned on the expandable member, and the receptacle can engage the lead body (e.g., a distal tip of the lead body) to secure the lead body to the expandable member. In some embodiments, the receptacle can be configured to release the lead body (e.g., by twisting the lead body relative to the receptacle), such that the lead body may be removed from the vasculature when not in use.

Alternatively or additionally, the one or more engagement members can include a spring-loaded pin housed within the receptacle. The spring-loaded pin can be configured to engage a slot of the lead body to releasably couple the lead body to the expandable member. For instance, the spring-loaded pin can be configured to be inserted into the slot of the lead body, thereby coupling the lead body to the expandable member. In some implementations, the lead body may be released from the expandable member through the use of a stylet, e.g., the stylet may be inserted into a lumen of the lead body and pressed against the spring-loaded pin to release the spring-loaded pin from the slot of the lead body.

In some embodiments, the one or more engagement members comprises one or more sutures (e.g., bioabsorbable sutures) configured to releasably couple the lead body to the expandable member. For instance, the sutures can be coupled to the lead body and threaded through one or more openings of the expandable member. Optionally, the sutures may be coupled to the lead body via an attachment positioned on the lead body. In some implementations, withdrawing the lead body relative to the expandable member can cause the sutures to break (e.g., tear), thereby decoupling the lead body from the expandable member.

The systems of the present technology can provide many advantages compared to conventional devices for delivering electrical current to stimulation sites from within the vasculature. For instance, the detachment mechanism(s) of the present technology can enable the removal of the lead body from bodily lumens when therapy is inactive while leaving the expandable member in place. In contrast with traditional medical implants that are permanent or semi-permanent, the removal of the lead body can prevent the risk of thrombosis, blood flow blockage, electrical hardware deterioration, device failure, etc. Allowing removal of the lead body without disturbing the expandable member also reduces the risk of damage to the treatment site as compared to conventional approaches that remove the entire assembly, including an expandable member, from the vessel. In addition, some treatments may benefit from leaving the expandable member in the vessel, e.g., to provide structural support to the vessel, even after therapy. Further, the present technology can provide minimally-invasive stimulation delivery that increases the number of accessible stimulation targets that would otherwise require invasive implantation. Moreover, the present technology can provide for the re-introduction of the lead body and secure engagement of the lead body at a treatment site, thus improving treatment precision and repeatability.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

### I. Overview of Example Treatment Systems and Devices

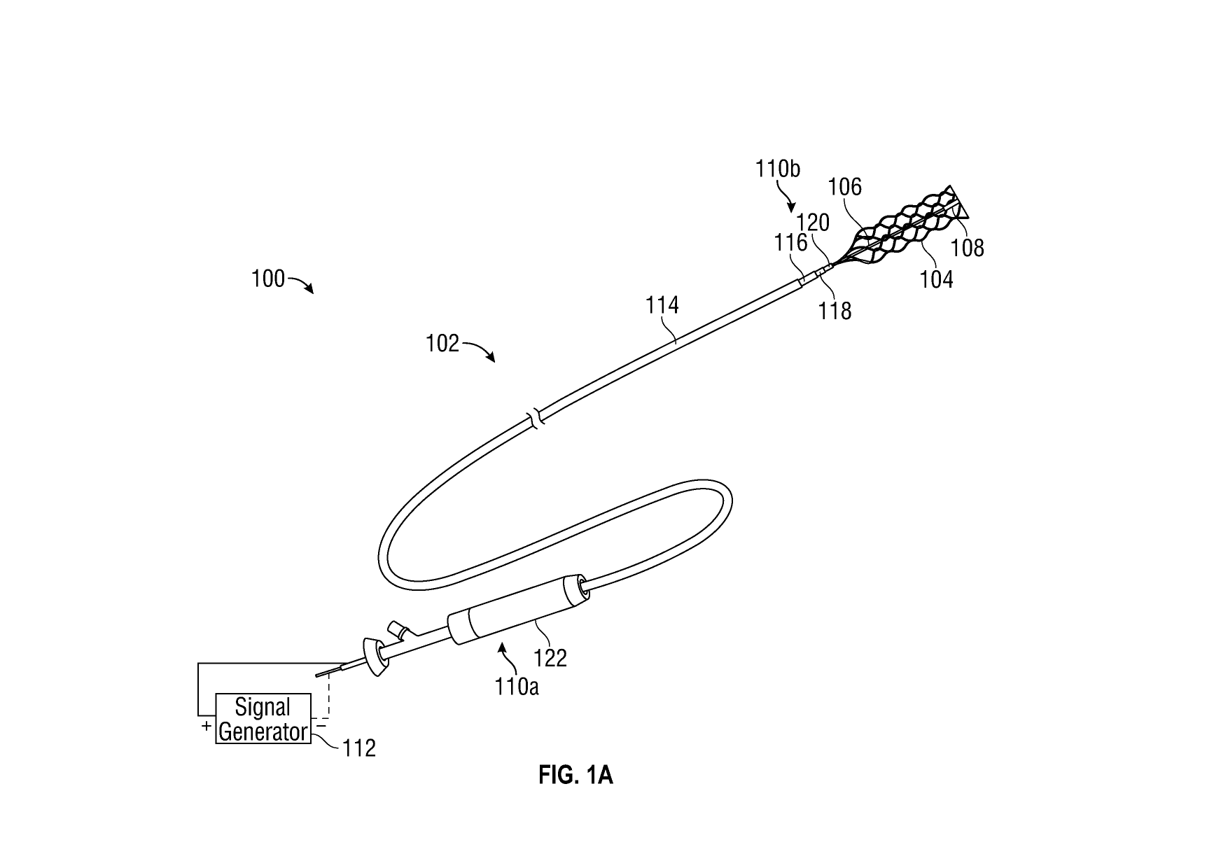

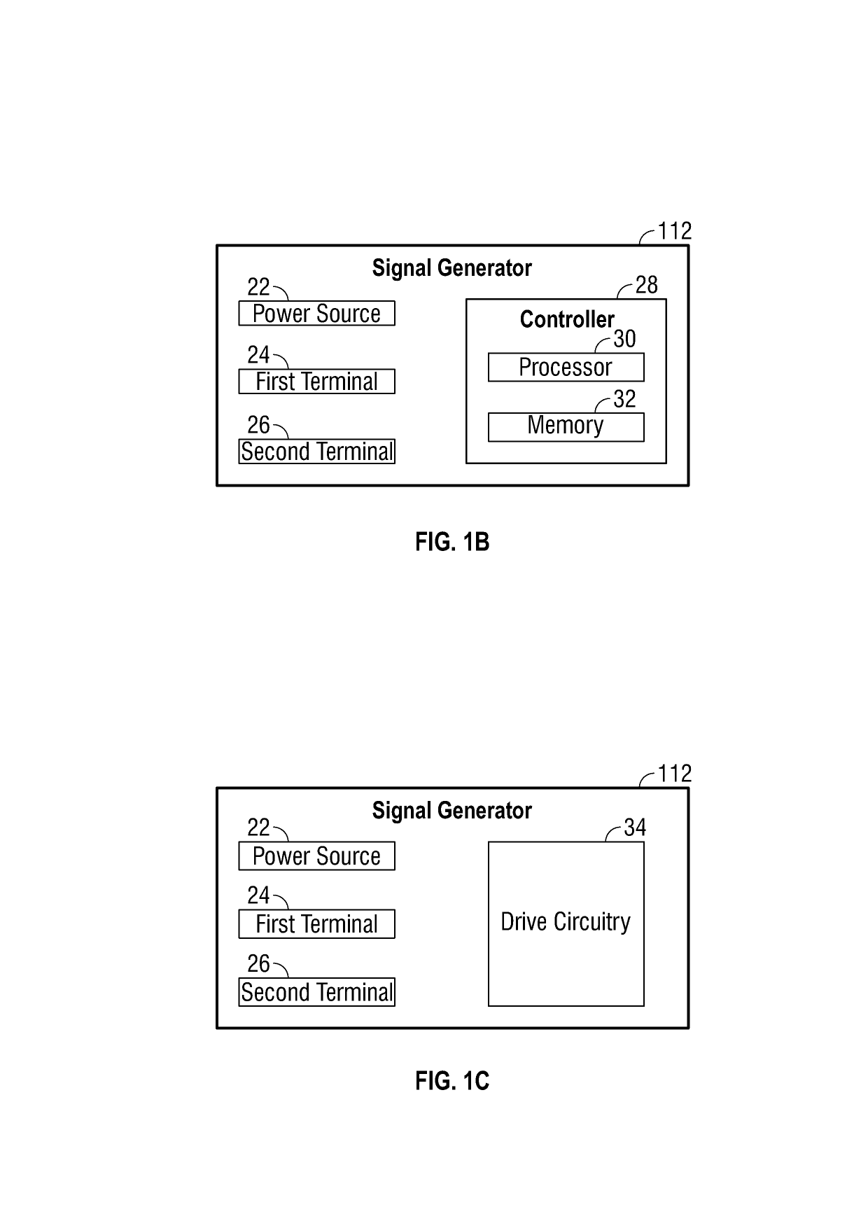

FIG. 1A illustrates a view of an electrical stimulation treatment system 100 in accordance with embodiments of the present technology. As shown in FIG. 1, the treatment system 100 can include a medical device assembly 102 comprising an expandable member 104, a lead body 106, and one or more engagement members 108. A proximal portion 110a of the medical device assembly 102 can be configured to be coupled to a signal generator 112 and a distal portion 110b of the medical device assembly 102 can be configured to be intravascularly positioned within a blood vessel (such as an intracranial blood vessel) at a treatment site, e.g., at or proximate a neural target. The medical device assembly 102 can further include a first catheter 114 (e.g., a balloon guide catheter), a second catheter 116 (e.g., a distal access catheter) configured to be slidable disposed within a lumen of the first catheter 114, a third catheter 118 (such as a microcatheter) configured to be slidably disposed within a lumen of the second catheter 116, and a core member 120 comprising the lead body 106, the core member 120 configured to be slidably disposed within a lumen of the third catheter 118. In some embodiments, the medical device assembly 102 does not include the second catheter 116. The medical device assembly 102 can also include a handle 122, which provides proximal access to one or more of the core member 120 (e.g., the lead body 106 of the core member 120), expandable member 104, first catheter 114, second catheter 116, or third catheter 118. The signal generator 112 may be coupled to a proximal portion of one or more of the core member 120 (e.g., at the lead body 106), first catheter 114, and/or third catheter 118 to provide an electrical current to the distal portion 110b of the medical device assembly 102, as described in more detail below.

With continued reference to FIG. 1A, in some examples, the treatment system 100 can be configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a tubular member, due at least in part to the tortuous pathway (e.g., comprising relatively sharp twists or turns) through the vasculature to reach these tissue sites. As such, the core member 120 may be structurally configured to be relatively flexible, pushable, and relatively kink- and buckle-resistant, so that it may resist buckling when a pushing force is applied to a relatively proximal section of the core member 120 to advance it distally through vasculature, and so that it may resist kinking when traversing around a tight turn in the vasculature. In some examples, the core member 120 is configured to substantially conform to the curvature of the vasculature. As an example, the lead body 106 of the core member 120 can have a column strength and flexibility that allow at least a distal portion of the lead body 106 to be navigated from a femoral artery, through the aorta of the patient, and into the intracranial vascular system of the patient, e.g., to reach a relatively distal treatment site.

Further, the catheters 114, 116, and 118 can each be formed as a generally tubular member extending along and about a central axis. The catheters 114, 116, and 118 can each be configured to accommodate the lead body 106 during insertion and/or removal of the lead body 106 through bodily lumens. In some embodiments, the third catheter 118 can be constructed to track over a conventional guidewire in the cervical anatomy and into the cerebral vessels associated with the brain and may also be chosen according to several standard designs that are generally available. Accordingly, the third catheter 118 can have a length that is at least 125 cm long, and more particularly may be between about 125 cm and about 175 cm long. Other designs and dimensions are contemplated.

The second catheter 116 can be sized and configured to slidably receive the third catheter 118 therethrough. The first catheter 114 can be sized and configured to slidably receive both the second catheter 116 and the third catheter 118 therethrough. In some embodiments, the first catheter 114 is a balloon guide catheter having an inflatable balloon or other expandable member surrounding the catheter shaft at or near its distal end. In operation, the first catheter 114 can first be advanced through a vessel and then its balloon can be expanded to anchor the first catheter 114 in place and/or arrest blood flow from areas proximal of the balloon. Next, the second catheter 116 can be advanced through the first catheter 114 until its distal end extends distally beyond the distal end of the first catheter 114. The second catheter 116 can be positioned such that its distal end is adjacent a treatment site (e.g., a site of a blood clot within the vessel). The third catheter 118 may then be advanced through the second catheter 116 until its distal end extends distally beyond the distal end of the second catheter 116. The expandable member 104 and/or lead body 106 may then be advanced through the third catheter 118 for delivery to the treatment site.

According to some embodiments, the bodies of the catheters 114, 116, and 118 can be made from various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters or an adjacent surface with a hydrophilic material such as polyvinylpyrrolidone (PVP) or some other plastic coating. Additionally, either surface can be coated with various combinations of different materials, depending upon the desired results.

Although primarily described as being used to reach relatively distal vasculature sites, the treatment system 100 may also be configured to be used with other target tissue sites. For example, the treatment system 100 may be used to access tissue sites throughout the coronary and peripheral vasculature, the gastrointestinal tract, the urethra, ureters, fallopian tubes, veins and other body lumens. For instance, target locations (e.g., the vagus nerve) may be accessed through peripheral veins (e.g., the jugular vein). In some examples, the treatment system 100 may be advanced through the jugular vein toward the vagus nerve which may advantageously improve device delivery over traditional nerve cuff leads that utilize invasive surgical procedures (e.g., nerve separation from the blood supply and wrapping of the nerve cuff around the vagus nerve).

According to some embodiments, the signal generator 112 can include an electrical generator configured to output medically useful electric current. FIGS. 1B and 1C are schematic views of different embodiments of the signal generator 112. With reference to FIG. 1B, the signal generator 112 can include a power source 22, a first terminal 24, a second terminal 26, and a controller 28. The controller 28 includes a processor 30 coupled to a memory 32 that stores instructions (e.g., in the form of software, code or program instructions executable by the processor or controller) for causing the power source 22 to deliver electric current according to certain parameters provided by the software, code, etc. The power source 22 of the signal generator 112 may include a direct current power supply, an alternating current power supply, and/or a power supply switchable between a direct current and an alternating current. The signal generator 112 can include a suitable controller that can be used to control various parameters of the energy output by the power source or generator, such as intensity, amplitude, duration, frequency, duty cycle, and polarity. For example, the signal generator 112 can provide a voltage of about 0.1 volts to about 10 volts and a current of about 0.1 mA to about 10 mA.

FIG. 1C illustrates another embodiment of the signal generator 112, in which the controller 28 of FIG. 1B is replaced with drive circuitry 34. In this embodiment, the signal generator 112 can include hardwired circuit elements to provide the desired waveform delivery rather than a software-based generator of FIG. 1B. The drive circuitry 34 can include, for example, analog circuit elements (e.g., resistors, diodes, switches, etc.) that are configured to cause the power source 22 to deliver electric current via the first and second terminals 24, 26 according to the desired parameters. For example, the drive circuitry 34 can be configured to cause the power source 22 to deliver periodic waveforms via the first and second terminals 24, 26.

As noted above, the signal generator 112 may be coupled to a proximal portion of the core member 120, and/or a proximal portion of the third catheter 118, the second catheter 116, and/or first catheter 114 to provide an electric current to the medical device assembly 102. For example, in some embodiments, both terminals of the signal generator 112 are coupled to the core member 120 (e.g., at the lead body 106) such that the core member 120 functions as both a delivery electrode or conductive path (i.e., transmitting current from the signal generator 112 to the treatment site) and a return electrode or conductive path (i.e., transmitting current from the treatment site to the signal generator 112). In other embodiments, the return electrode can be separate from the core member 120. For example, the return electrode can be carried by one or more of the third catheter 118, the second catheter 116, and/or first catheter 114. In some embodiments, the return electrode can be provided via one or more external electrodes, such as a needle puncturing the patient or a grounding pad applied to the patient’s skin. In some embodiments, the return electrode can be an insulated guide wire having an exposed, electrically conductive portion at its distal end.

In some embodiments, one or more electrodes can be carried by, coupled to, and/or mounted on the lead body 106 and/or expandable member 104 (or the electrodes can comprise conductive elements or surfaces other than radiopaque elements/markers (if any)). The electrodes can optionally take the form of radiopaque elements or markers affixed to a portion of the expandable member 104 and/or the lead body 106, and can be arranged so as to provide and/or improve electrical current delivery to the surrounding tissue. In some embodiments, current can be delivered to the electrodes over a plurality of corresponding electrical leads extending between the signal generator 112 and the electrodes affixed to the lead body 106 and/or expandable member 104. As an example, the lead body 106 can include a plurality of contact pads and each of the contact pads can be connected to a respective electrical lead housed in the lead body 106. Alternatively or in combination, the expandable member 104 can include a plurality of contact pads, and each of the contact pads can be connected to a respective electrical lead housed in the lead body 106 or elsewhere. The electrodes can comprise delivery electrodes as well as one or more return electrodes, which can likewise be coupled to or formed on the expandable member 104, lead body 106, or elsewhere (e.g., as an external electrode).

### II.Detachable Stent-Lead Combination

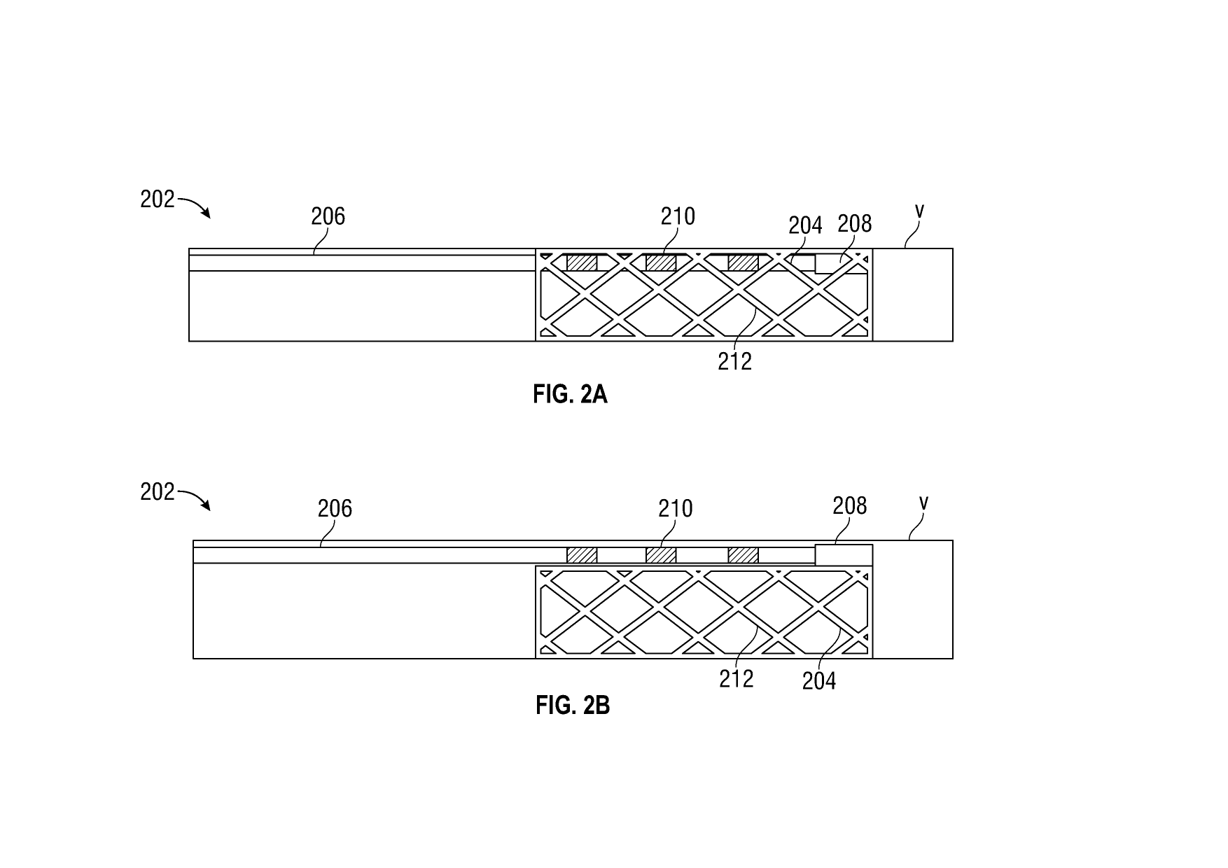

FIGS. 2A and 2B are close-up side views of a medical device assembly 202, in accordance with embodiments of the present technology. The medical device assembly 202 can be generally similar to the medical device assembly 102 of the treatment system 100 of FIG. 1. For instance, the medical device assembly 202 can include an expandable member 204 and a lead body 206 releasably coupled to the expandable member 204 via one or more engagement members 208. The one or more engagement members 208 can be positioned in a lumen of the expandable member 204 (e.g., as depicted in FIG. 2A), or on a radially outer side of the expandable member 204 (e.g., as depicted in FIG. 2B). Optionally, in some implementations, the medical device assembly 202 can include multiple expandable members 204 and/or multiple lead bodies 206 with a variety of engagement member 208 positions.

Referring to FIGS. 2A and 2B together, in some embodiments, the expandable member 204 is configured to be deployed at a treatment site within a blood vessel V. The expandable member 204 can have a low-profile, constrained or compressed configuration (not shown) for intravascular delivery to the treatment site and an expanded configuration (as depicted) for anchoring the medical device assembly 202 at the treatment site, supporting and/or expanding the surrounding vessel wall(s), and/or for restoring blood flow at the treatment site. For instance, the expandable member 204 can be configured to contact the surrounding vessel wall(s) such that the expandable member 204 is resistant to axial movement.

In some embodiments, the expandable member 204 includes an open cell framework or body 212. In some embodiments, the body 212 of the expandable member 204 can be generally tubular (e.g., cylindrical), and the proximal portion and/or the distal portion can include tapered regions (e.g., during delivery and/or after implantation). In some embodiments, the expandable member 204 is a mesh structure (e.g., a braid, a stent, etc.) formed of a superelastic material (e.g., Nitinol) or other resilient or self-expanding material configured to self-expand when introduced at the treatment site. The one or more engagement members 208 may be attached to the body 212 and/or the mesh structure as applicable.

In various embodiments, the expandable member 204 can take any number of forms, for example an anchoring device, a thrombectomy device, or other suitable medical device. For example, in some embodiments the expandable member 204 may be a stent and/or stent retriever, such as Medtronic's Solitaire^{TM} Revascularization Device, Stryker Neurovascular's Trevo® ProVue^{TM} Stentriever, or other suitable devices. In some embodiments, the expandable member 204 may be a coiled wire, a weave, and/or a braid formed of a plurality of braided filaments. Examples of suitable expandable members 204 include any of those disclosed in U.S. Patent No. 7,300,458, filed November 5, 2007, U.S. Patent No. 8,940,003, filed November 22, 2010, U.S. Patent No. 9,039,749, filed October 1, 2010, and U.S. Patent No. 8,066,757, filed December 28, 2010, each of which is incorporated by reference herein in its entirety.

The lead body 206 can be configured to facilitate electrical stimulation of surrounding tissue at the treatment site via one or more electrodes 210. For instance, each electrode 210 can be disposed on the lead body 206 and take the form of electrically conductive members or surfaces (e.g., coils, tubes, bands, plates, traces, or any other suitable structure that is electrically conductive). Exemplary materials for the electrodes 210 include copper, stainless steel, nitinol, platinum, gold, iridium, tantalum, alloys thereof, or any other suitable materials that are electrically conductive.

The electrodes 210 can each be coupled to a respective electrical lead (not depicted) that may extend alongside and/or be housed in the lead body 206. For instance, the electrical leads can be bundled together or otherwise grouped together in a lead bundle assembly that extends through the lead body 206. The bundle assembly can couple at a proximal end portion to a signal generator (e.g., the signal generator 112 of the treatment system 100 of FIG. 1A), with each individual lead being electrically coupled to the signal generator to carry current to a respective electrode 210. Alternatively or in combination, in some embodiments, any subset of the electrodes 210 may share electrical connection via one or more leads. For example, a lead may extend between two electrodes 210, thereby placing those two electrodes in electrical communication with another as well as the generator or other current source, when coupled thereto.

In some embodiments, a first subset of the electrodes 210 can be electrically coupled to the positive terminal of the signal generator 112 via their respective leads 216, and accordingly serve as delivery electrodes. Meanwhile, a second subset of the electrodes 210 can be electrically coupled to the negative terminal of the signal generator 112 via their respective leads and accordingly serve as return electrodes. In some embodiments, some or all of the delivery electrodes 210 can be in electrical communication with the blood vessel V without contacting the expandable member 204. For instance, the delivery electrodes 210 may be positioned within one or more openings of the expandable member 204 such that the delivery electrodes 210 protrude through the expandable member 204 toward the vessel wall(s). Optionally, the expandable member 204 may be electrically insulated from the delivery electrodes 210. For instance, one or more regions of the expandable member 204 can be coated with an insulative material such that current carried by the delivery electrodes 210 within the expandable member 204 will not be carried by the surface of the expandable member 204 in the coated regions.

Alternatively, some or all of the delivery electrodes 210 can be in electrical communication with the expandable member 204 which may itself be electrically conductive. When some or all of the delivery electrodes 210 are in electrical communication with the (electrically conductive) body of the expandable member 204, the positive/delivery lead (e.g., a single such lead) can be electrically coupled to the expandable member 204, e.g., at or near the proximal end thereof, and thereby in electrical communication with some or all of the delivery electrodes 210. As such, current carried by the delivery electrodes 210 can flow into the expandable member 204, thereby generating a positive charge along at least a portion of the expandable member 204 (as well as any delivery electrodes 210 coupled to the expandable member 204).

In some embodiments, the return electrodes 210 can be carried by the expandable member 204 but be electrically insulated from the body of the expandable member 204. For example, the return electrodes 210 can be mounted over a portion of the expandable member 204 with an electrically insulating material disposed therebetween such that current carried by a return electrode 210 does not pass to the body of the expandable member 204, but instead passes through the corresponding lead coupled to the return electrode 210. In some embodiments, the return electrodes 210 can be in electrical communication with at least a portion of the expandable member 204.

Instead of or in addition to the return electrodes 210 carried by the expandable member 204, the return electrode(s) can assume a variety of different configurations. For example, in some embodiments, the return electrode is an external electrode, such as a needle or grounding pad that is applied to a patient’s skin. The needle or grounding pad can be coupled via one or more leads to the signal generator 112 to complete the electrical circuit. In some embodiments, the return electrode is carried by a surrounding catheter (e.g., third catheter 118, second catheter 116, and/or first catheter 114). In some embodiments, the return electrode can be an insulated guide wire having an exposed, electrically conductive portion at its distal end, or an exposed, electrically conductive portion of the core member 120 near its distal end.

As noted previously, the lead body 206 and the expandable member 204 can be releasably coupled via one or more engagement members 208. For instance, an engagement member 208 can be positioned on the expandable member 204 and be configured to receive the lead body 206. Alternatively, the engagement member 208 can be positioned on the lead body 206 and be configured to receive the expandable member 204.

In some embodiments, the engagement member 208 comprises a receptacle 208. The receptacle 208 can be coupled to the expandable member 204. For instance, in some embodiments, the receptacle 208 is coupled to the expandable member 204 via one or more of adhesives, bonding, threads, fasteners, welding, etc. Alternatively or in combination, the receptacle 208 can be integrally formed with the expandable member 204. In some embodiments, the receptacle 208 is positioned proximate to only a distal portion of the expandable member 204. Alternatively, the receptacle 208 can be positioned proximate to (e.g., span) an entire length of the expandable member 204, or less than the length of the expandable member 204, e.g., no more than 80%, 70%, 60%, 50%, 40%, 30%, 20%, 10%, 5%, or 3% of the length of the expandable member 204. Further, the receptacle 208 can be additionally or alternatively positioned proximate to a proximal portion of the expandable member 204 or an intermediate portion of the expandable member 204.

In some embodiments, as shown in FIG. 2A, the receptacle 208 is positioned within a lumen of the expandable member 204. For instance, the receptacle 208 can be positioned in the lumen of the expandable member 204 proximate to a distal portion of the expandable member 204. In other embodiments, as shown in FIG. 2B, the receptacle 208 is positioned external to the lumen of the expandable member 204. For instance, the receptacle 208 can be positioned on a radially outer side of the expandable member 204. The illustrated positions of the receptacle 208 are not limiting, and the receptacle 208 may be positioned anywhere within and/or around the expandable member 204 as desired. Further, the engagement members 208 may comprise a plurality of receptacles 208, as will be explored further herein.

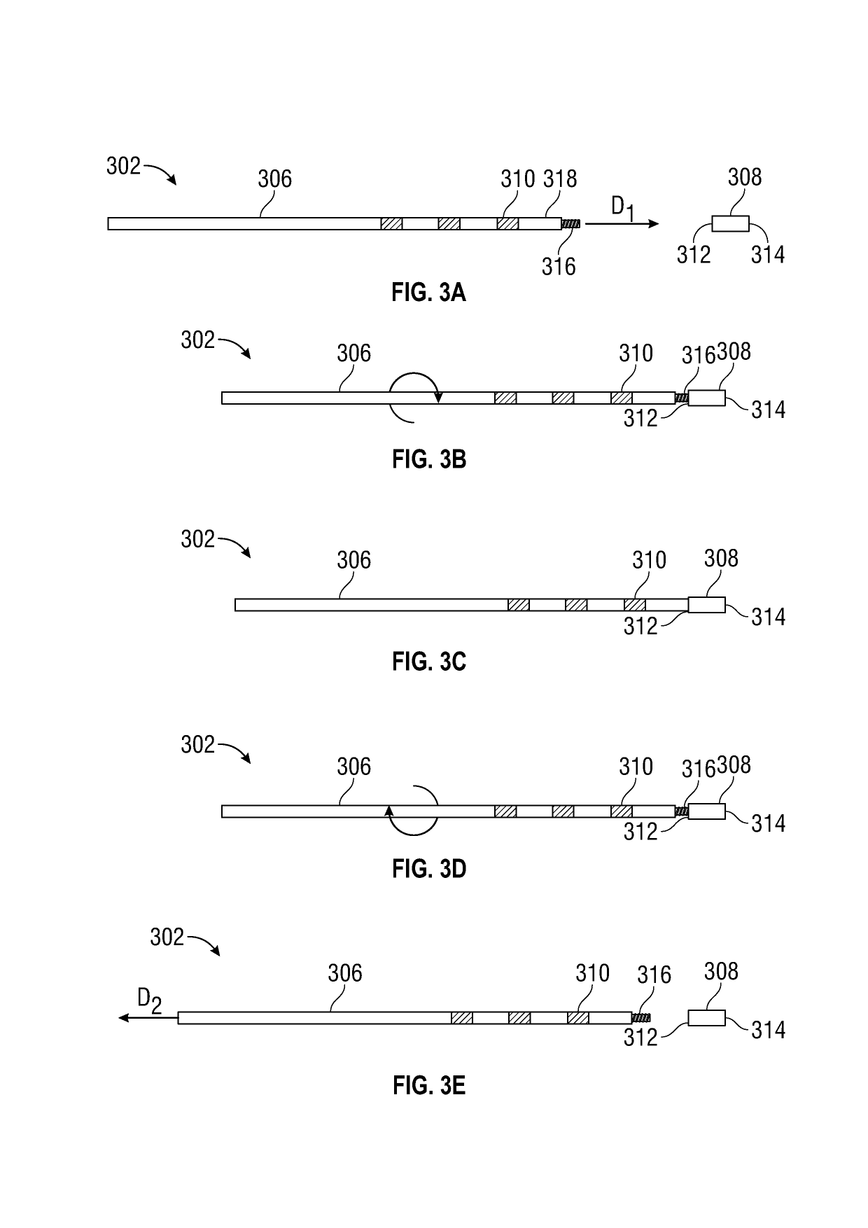

FIGS. 3A-3E illustrate side views of a medical device assembly 302 in various configurations, in accordance with embodiments of the present technology. Specifically, FIG. 3A is a schematic illustration of the medical device assembly 302 in a disengaged configuration, FIG. 3B is a schematic illustration of the medical device assembly 302 in a partially-engaged configuration, FIG. 3C is a schematic illustration of the medical device assembly 302 in an engaged configuration, FIG. 3D is another schematic illustration of the medical device assembly 302 in a partially-engaged configuration, and FIG. 3E is another schematic illustration of the medical device assembly 302 in a disengaged configuration.

Referring to FIGS. 3A-3E collectively, the medical device assembly 302 can be generally similar to the medical device assemblies described elsewhere herein, such as the medical device assembly 202 of FIG. 2. For instance, the medical device assembly 302 can include an expandable member (e.g., the expandable member 204 of FIG. 2) and a lead body 306 configured to be releasably coupled to the expandable member via one or more engagement members 308. In some embodiments, the expandable member is a stent configured to maintain an axial position within a vessel at a treatment site. For instance, the expandable member can include a mesh structure (e.g., a braid) configured to self-expand when introduced at the treatment site. Accordingly, the expandable member can be configured to be disposed at the treatment site for a prolonged period of time. The lead body 306 can include a plurality of electrodes 310 disposed thereon, the electrodes 310 configured to deliver electrical current to surrounding tissue. In some implementations, the lead body 306 can be coupled to a signal generator (e.g., the signal generator 112 of the treatment system 100 of FIG. 1) in a proximal region of the lead body 306 and to the electrodes 310 in a distal region of the lead body 306.

The engagement members 308 can be coupled to the expandable member at a variety of positions. In some embodiments, the engagement members 308 are coupled to the expandable member at a distal portion of the expandable member. Alternatively, the engagement members 308 can be coupled to the expandable member at a proximal or intermediate portion of the expandable member. The engagement members 308 can be affixed, welded, adhered, tied, etc. to the expandable member. Further, the engagement members 308 can be positioned within a lumen of the expandable member, or on a radially outer surface of the expandable member.

In some embodiments, the engagement members 308 includes a receptacle 308. The receptacle 308 can be configured to receive the lead body 306. The receptacle 308 can be generally cylindrical, rectangular, and/or any other suitable geometry for receiving the lead body 306. In some embodiments, the receptacle 308 includes an open end 312 and a closed end 314. The open end 312 of the receptacle 308 can be configured to mate with the lead body 306. For instance, the open end 312 of the receptacle 308 may include threading, grooves, teeth, frictional elements, protrusions, indentations, and/or other suitable features for engaging the lead body 306.

In some embodiments, the lead body 306 includes a distal tip 316. The distal tip 316 can extend from a distal end region 318 of the lead body 306. The distal tip 316 can be generally cylindrical, but may additionally or alternatively include other geometries and/or features, such as barbs, tines, hooks, pins, cones, etc. In some embodiments, the distal tip 316 includes a threaded tip 316. For instance, the threaded tip 316 may include helical threads and/or ridges. The threaded tip 316 can be configured to mate with the receptacle 308. For instance, the threaded tip 316 may be fastened into one or more grooves of the receptacle 308.

As shown in FIG. 3A, the lead body 306 can be advanced toward the receptacle 308 in the direction D₁. In some embodiments, the lead body 306 is advanced with the threaded tip 316 facing the open end 312 of the receptacle 308. Once the threaded tip 316 contacts the open end 312, the lead body 306 can be rotated (e.g., in a clockwise direction) while maintaining a distal axial force (e.g., in the direction D₁), causing the threaded tip 316 to be inserted into and secured within the receptacle 308 (shown in a partially-engaged configuration in FIG. 3B and in a fully-engaged configuration in FIG. 3C). In the engaged configuration of FIG. 3C, the lead body 306 and the expandable member are immovable or substantially immovable relative to one another axially. In some embodiments, the medical device assembly 302 is advanced to a treatment site in the engaged configuration. For instance, the lead body 306 can be used to at least partially support the distal advancement of the expandable member 304, e.g., by transferring forces distally. As discussed elsewhere herein, the lead body 306 and/or the medical device assembly 302 can be manipulated (e.g., advanced, retracted, or rotated) using a handle, such as the handle 122 of the treatment system 100 of FIG. 1.

To disengage the lead body 306 from the expandable member, the lead body 306 can be rotated again (e.g., in a counter-clockwise direction) while applying a proximal axial force (e.g., in the direction D₂), causing the threaded tip 316 to be retracted and removed from the receptacle 308 (shown in a partially-engaged configuration in FIG. 3D and a disengaged configuration in FIG. 3E). After the threaded tip 316, and thereby the lead body 306, has been removed from the receptacle 308, the lead body 306 can be proximally retracted. For instance, the lead body 306 can be retracted in the direction D₂ and removed from the vasculature. In some embodiments, the lead body 306 can be re-introduced into the vasculature and configured to re-engage the expandable member 304 using the same and/or a similar engagement process to that described above with respect to FIGS. 3A-3C. The reintroduction and withdrawal of the lead body 306 from the treatment site can be repeated as necessary, such as over a multi-session treatment.

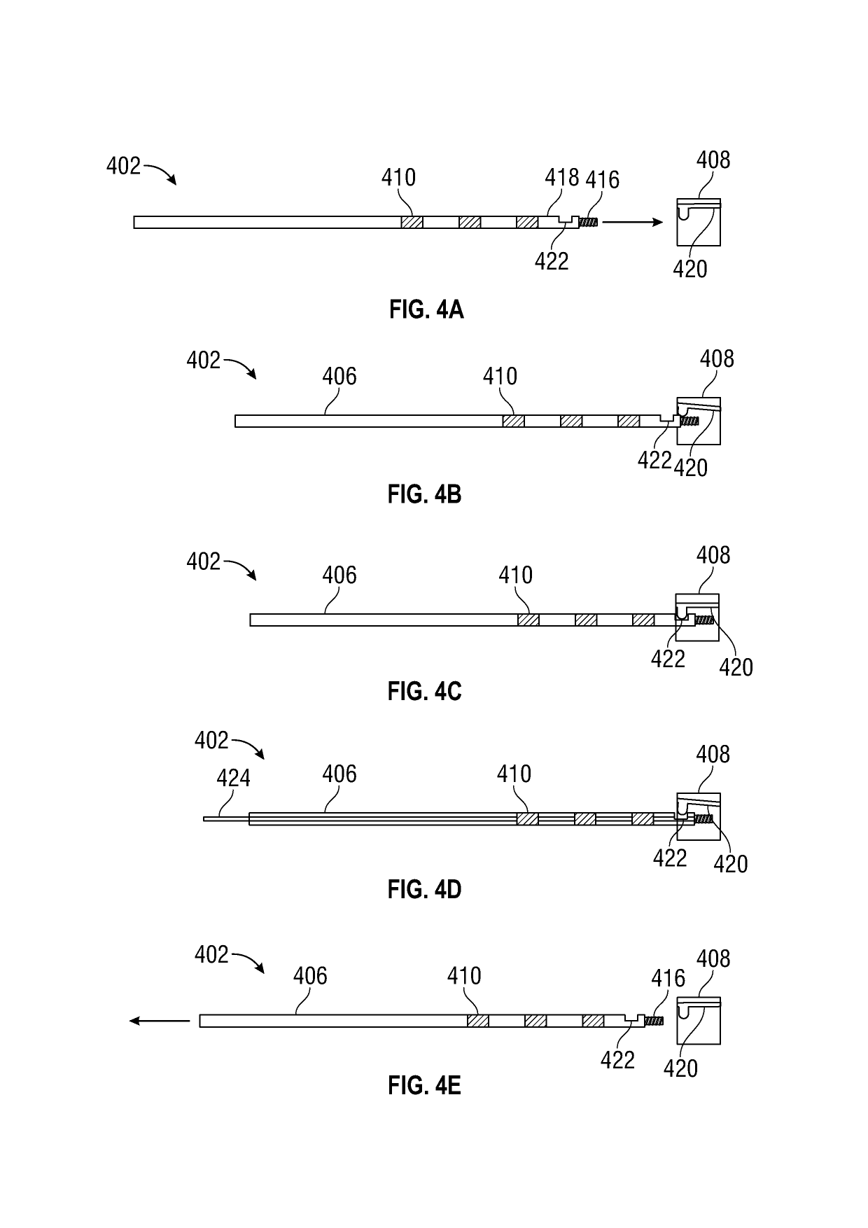

FIGS. 4A-4E illustrate side views of a medical device assembly 402 in various configurations, in accordance with embodiments of the present technology. Specifically, FIG. 4A is a schematic illustration of the medical device assembly 402 in a disengaged configuration, FIG. 4B is a schematic illustration of the medical device assembly 402 in a partially-engaged configuration, FIG. 4C is a schematic illustration of the medical device assembly 402 in an engaged configuration, FIG. 4D is a another schematic illustration of the medical device assembly 402 in a partially-engaged configuration, and FIG. 4E is another schematic illustration of the medical device assembly 402 in a disengaged configuration.

Referring to FIG. 4A–4E collectively, the medical device assembly 402 can be generally similar to the medical device assemblies described elsewhere herein, such as the medical device assembly 202 of FIG. 2. For instance, the medical device assembly 402 can include an expandable member (not depicted) and a lead body 406 configured to be releasably coupled to the expandable member via one or more engagement members 408. In some embodiments, the expandable member is a stent configured to maintain an axial position within a vessel at a treatment site. For instance, the expandable member can include a mesh structure (e.g., a tubular braid, laser-cut stent, etc.) configured to self-expand when introduced at the treatment site. Accordingly, the expandable member can be configured to be disposed at the treatment site for a prolonged period of time. The lead body 406 can include a plurality of electrodes 410 disposed thereon, the electrodes 410 configured to deliver electrical current to surrounding tissue. In some implementations, the lead body 406 can be coupled to a signal generator (e.g., the signal generator 112 of the treatment system 100 of FIG. 1) in a proximal region of the lead body 406 and to the electrodes 410 in a distal region of the lead body 406.

The engagement members 408 can be generally similar to the engagement members discussed elsewhere herein, such as the engagement members 308 of the medical device assembly 302 of FIG. 3. For instance, the engagement members 408 can be positioned at a variety of locations such as at proximal, distal, internal, and/or external portions of the expandable member. In some embodiments, the engagement members 408 includes a receptacle 408. The receptacle 408 can be configured to receive the lead body 406, and the receptacle 408 can include an open end 412 and a closed end 414. Optionally, the receptacle 408 can be configured to receive a distal tip 416 protruding from a distal end region 418 of the lead body 406.

In some embodiments, the receptacle 408 includes a pin 420. The pin 420 can be an elongate member projecting from a lumen of the receptacle 408. In some embodiments, the pin 420 is a spring-loaded pin 420. For instance, the pin 420 may have an internal spring force configured to bias the pin 420 toward the lead body 406 when the lead body 406 is housed within the receptacle 408. For instance, the pin 420 may be biased downwards. Alternatively or additionally, the pin 420 may be affected by other internal and/or external forces such as pressure, stress, suction, gravity, etc.

In some embodiments, the pin 420 is received by a slot 422 of the lead body 406. For instance, the slot 422 can be positioned near the distal end region 418 of the lead body 406. The slot 422 can include an opening sized to receive at least a portion of the pin 420 to secure the pin 420 in-place. In some embodiments, the slot 422 is rectangular, circular, or any other geometry shaped to accommodate the portion of the pin 420.

Referring now to FIG. 4A, the lead body 406 can be advanced toward the receptacle 408 in the direction D₁. In some embodiments, the lead body 406 is advanced with the distal end region 418 facing the open end 412 of the receptacle. Once the distal end region 418 abuts the open end 412 of the receptacle, the distal end region 418 may at least partially displace the pin 420. For instance, the distal end region 418 may cause the pin 420 to temporarily bend upwardly into an unrelaxed state (e.g., substantial amount of potential energy), as depicted in FIG. 4B. Upon the further advancement of the lead body 406, the pin 420 may return to its relaxed state (e.g., zero or little potential energy) by sliding into the slot 422 of the lead body 406, as depicted in FIG. 4C. Optionally, when the lead body 406 includes the distal tip 416, the distal tip 416 can be secured into the receptacle 408 as discussed above with respect to the medical device assembly 302 of FIGS. 3A–3E.

As shown in FIG. 4D, the spring-loaded pin 420 can be released from the slot 422 using a stylet 424. The stylet 424 can be any elongate member configured to be received by a lumen of the lead body 406. In some embodiments, the stylet 424 is configured to contact the pin 420 to displace the pin 420 from the slot 422 of the lead body 406, thereby decoupling the lead body 406 from the expandable member 404. The lead body 406 can then be proximally retracted and removed from the vessel, as shown in FIG. 4E. In some embodiments, the lead body 406 can be re-introduced into the vasculature and configured to re-engage the expandable member 404 using the same and/or a similar engagement process to that described above with respect to FIG. 4A–4C. The reintroduction and withdrawal of the lead body 406 from the treatment site can be repeated as necessary, such as over a multi-session treatment.

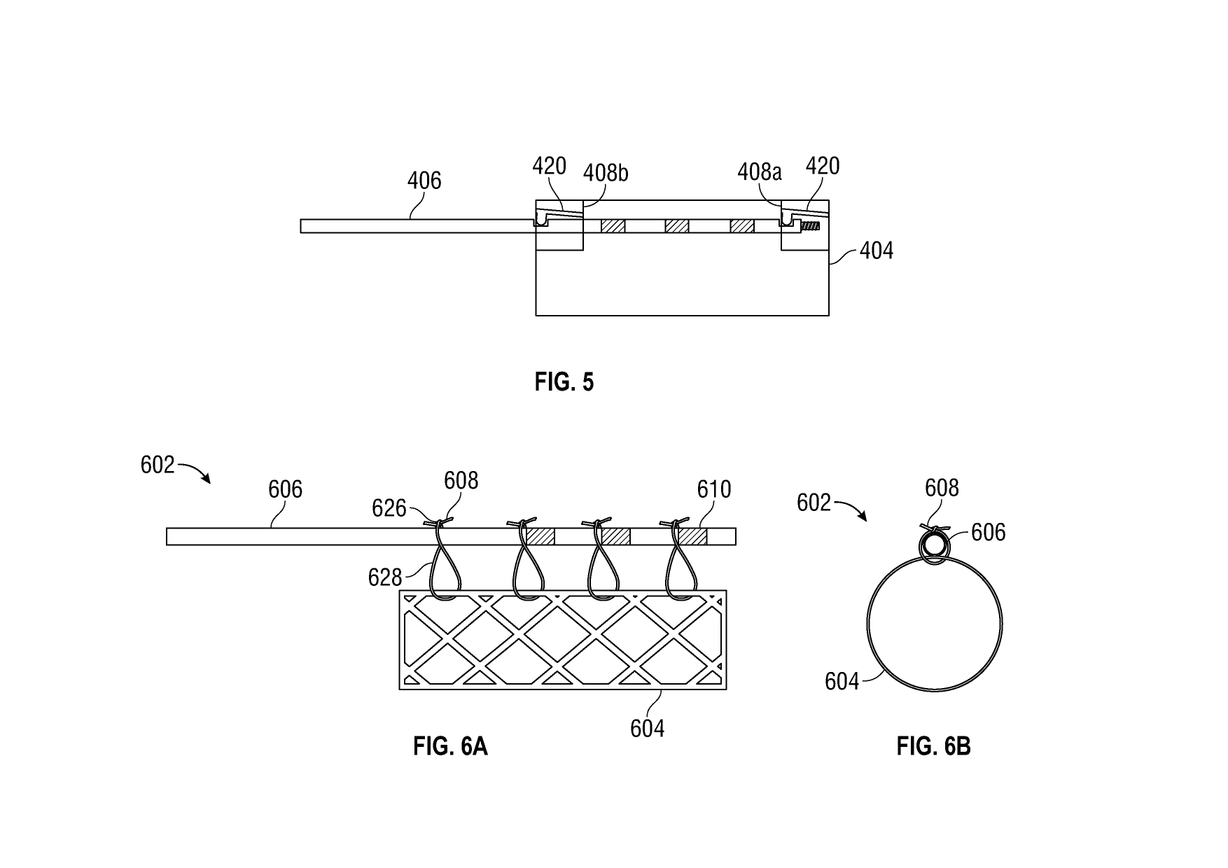

Optionally, the one or more engagement members 408 may include a plurality of receptacles 408. The plurality of receptacles 408 can include any number of receptacles 408, such as 2, 3, 4, 5, 6, 7, 8, 9, or 10 receptacles 408. For instance, as depicted in FIG. 5, a first receptacle 408a of the plurality of receptacles 408 can be positioned at a distal portion of the expandable member, and a second receptacle 408b of the plurality of receptacles 408 can be positioned at a proximal portion of the expandable member. Each of the receptacles 408 can include respective pins 420. The pins 420 may be configured to couple and decouple the lead body 406 and the expandable member 404 as discussed above with respect to FIG. 4A–4E.

FIGS. 6A and 6B illustrate a medical device assembly 602, in accordance with embodiments of the present technology. Specifically, FIG. 6A is a side view of a distal portion of the medical device assembly 602, and FIG. 6B is an end view of the medical device assembly 602. Referring to FIGS. 6A and 6B together, the medical device assembly 602 can be generally similar to any of the medical device assemblies discussed herein, such as the medical device assembly 202 of FIG. 2. For instance, the medical device assembly 602 can include an expandable member 604, a lead body 606, and one or more engagement members 608 configured to releasably couple the lead body 606 to the expandable member 604.

In some embodiments, the one or more engagement members 608 comprise sutures 608. The sutures 608 can be configured to contact one or more of the expandable member 604 or the lead body 606. In some embodiments, a first end region 626 of the sutures 608 is configured to be releasably coupled (e.g., tied, threaded) to the lead body 606 and a second end region 628 of the sutures 608 is configured to be releasably coupled (e.g., tied, threaded) to the expandable member 604. The sutures 608 can include bioabsorbable thread (e.g., polygactin, cat gut, poliglecaprone, etc.) and/or non-absorbable thread (e.g., silk, nylon, braided polyester, PFTE, stainless steel, etc.). Further, the sutures 608 can include monofilament and/or polyfilament materials. In some embodiments, the sutures 608 include at least 1, 2, 3, 4, 5, 6, 7, 8, 9, or 10 sutures 608. Optionally, the sutures 608 may include a coating, such as a bio-compatible coating.

The sutures 608 may be applied to the lead body 606 and the expandable member 604 prior to introduction of the medical device assembly 602 into the vasculature. For instance, the sutures 608 may be fastened such that the lead body 606 is secured to the expandable member 604 with minimal or little axial movement between them. The medical device assembly 602 can be advanced to the treatment site as discussed above with respect to the treatment system 100 of FIG. 1. For instance, the expandable member 604 can be allowed to expand at the treatment site, anchoring the medical device assembly 602 at the treatment site. In some embodiments, the lead body 606 can be decoupled from the expandable member 604 by proximally retracting the lead body 606 while the expandable member 604 is in an expanded configuration. The proximal retraction can cause the sutures 608 to break apart, tear, fragment, fracture, etc., allowing for the release of the lead body 606 from the expandable member 604. The lead body 606 can then be fully removed from the vessel as desired.

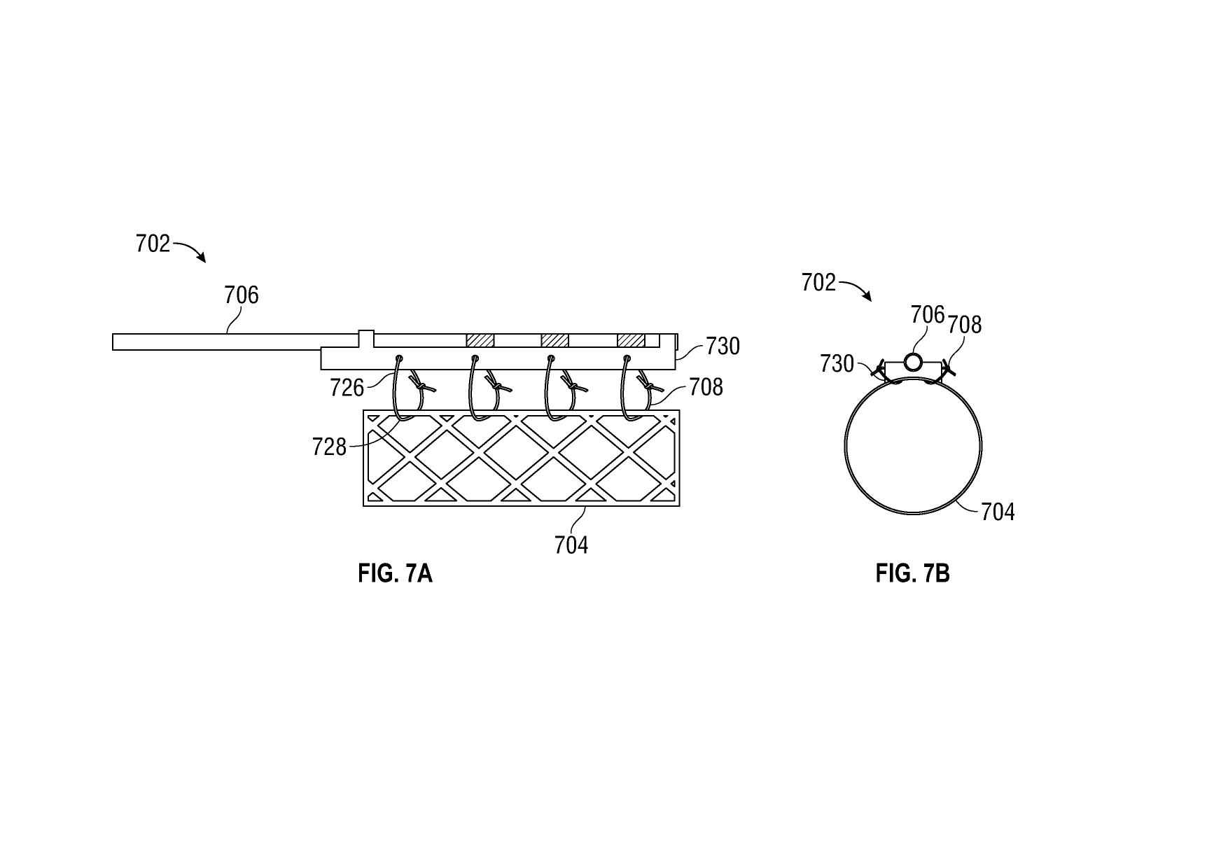

While the sutures 608 have been described as being directly coupled to the lead body 606 and the expandable member 604, in some embodiments, the sutures 608 are configured to be releasably coupled to an attachment positioned on one or more of the expandable member 604 or the lead body 606. Turning now to FIGS. 7A and 7B, a medical device assembly 702 can be generally similar to the medical device assembly 602 of FIG. 6, except for as described below. In the medical device assembly 702, sutures 708 are releasably coupled at a first end 726 to an attachment 730 positioned on the lead body 706 and at a second end 728 to the expandable member 704. In some embodiments, attaching the sutures 708 to the attachment 730 instead of directly to the lead body 706 may reduce the risk of damaging the lead body 706. Further, the attachment 730 may include a softer material than the lead body 706 such that the sutures 708 are more easily released from the attachment 730. For example, the attachment 730 may include biocompatible silicones and/or polyurethanes. In some embodiments, the attachment 730 includes a bioabsorbable pouch (e.g., monofilament knitted mesh). The attachment 730 can include any suitable geometry, such as rectangular, circular, oblong, etc. Further, the attachment 730 may include one or more flaps configured to receive and release the sutures 708. Alternatively or in addition, the attachment 730 may include thin wall regions where the sutures 708 are more easily removed.

In some embodiments, the attachment 730 and/or the lead body 706 include one or more gripping elements configured to assist in the positioning of the lead body 706 at the treatment site. For instance, the one or more gripping elements can include barbs, tines, grooves, ridges, etc. for accommodating one or more of the vessel wall(s), the expandable member 704, or other electrical hardware. The gripping elements may provide support to the lead body 706 at the treatment site, for example, when the sutures 708 have been absorbed or degraded.

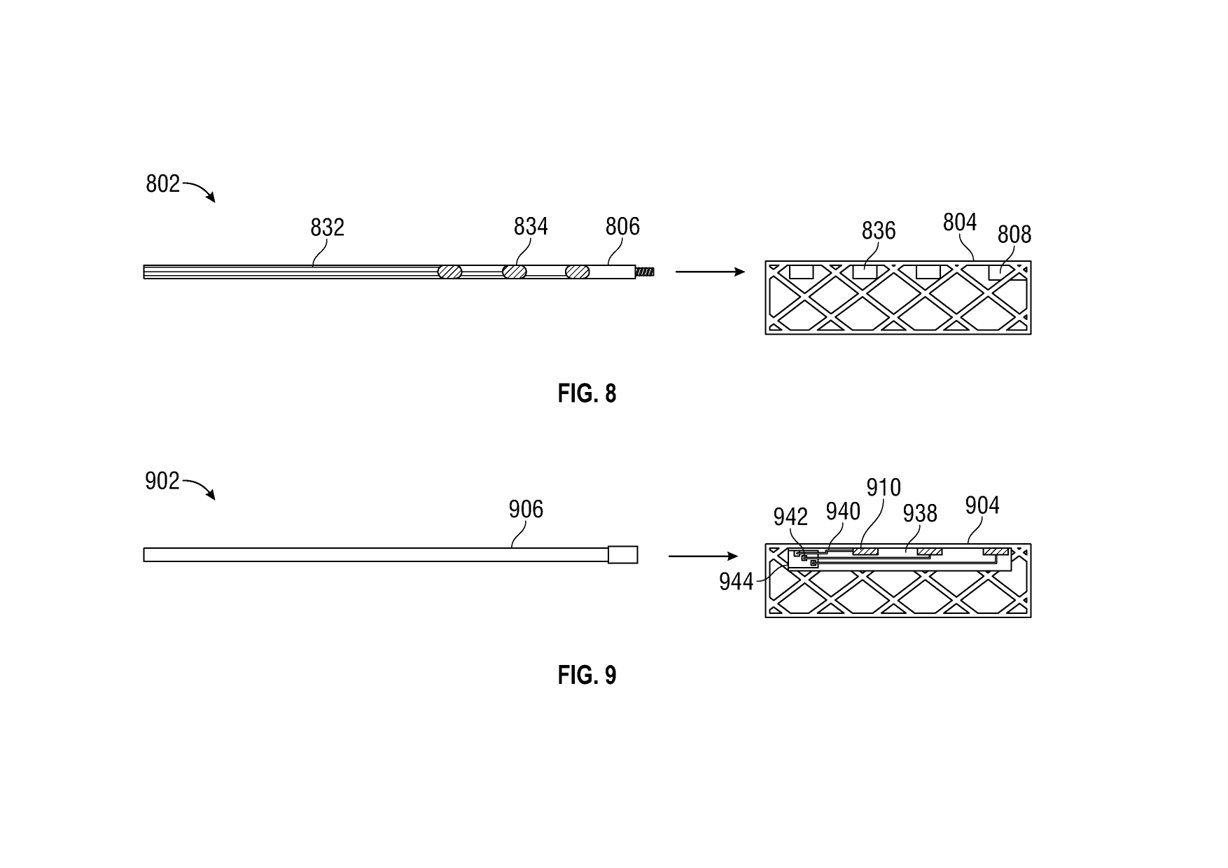

FIG. 8 illustrates a medical device assembly 802 in accordance with embodiments of the present technology. The medical device assembly 802 can be generally similar to any of the medical device assemblies discussed herein, except as described below. For instance, the medical device assembly 802 can include an expandable member 804 and a tubular member 806 configured to be releasably coupled with the expandable member 804 via one or more engagement members 808. The coupling of the expandable member 804 and the tubular member 806 can be the same or similar to any of the coupling features and/or processes discussed above with respect to FIG. 2A–7B.

In some embodiments, the tubular member 806 includes a plurality of lead wires 832. The lead wires 832 can each be coupled to a signal generator (e.g., the signal generator 112 of the treatment system 100 of FIG. 1) at a first (e.g., proximal) end of the lead wires 832. The lead wires 832 can be configured to carry current from the signal generator distally. For instance, in some embodiments, a second end of the lead wires 832 is in electrical communication with a plurality of contact pads 834 disposed on the tubular member 806. The contact pads 834 can be configured to transfer current from the lead wires 832 to one or more electrodes 836 disposed on or in the expandable member 804. For instance, the contact pads 834 may contact the electrodes 836 when the tubular member 806 is coupled to the expandable member 804 via the one or more engagement members 808. In some embodiments, the contact pads 834 directly overlap with the electrodes 836. In other embodiments, the contact pads 834 only partially overlap with the electrodes 836. The contact pads 834 may have a different surface geometry than the electrodes 836. For example, the contact pads 834 may have a smaller surface area than that of the electrodes 836.

In some embodiments, the contact pads 834 are omitted. For instance, the lead wires 832 of the tubular member 806 may be configured to directly contact the electrodes 836 of the expandable member 804. In such cases, the tubular member 806 may include a plurality of openings, and the lead wires 832 may be routed through the openings to contact the electrodes 836 when the tubular member 806 is coupled to the expandable member 804.

In some embodiments, the electrodes 836 are predisposed in the expandable member 804. For instance, the expandable member 804 can be manufactured to include the electrodes 836. Alternatively or in combination, the electrodes 836 can be initially coupled to the tubular member 806 and disposed on or in the expandable member 836 during disengagement of the tubular member 806 from the expandable member 804. For instance, decoupling the tubular member 806 and the expandable member 836 via the engagement members 808 can include separating the electrodes 836 from the tubular member 806. In some embodiments, separation is achieved by cutting the electrodes 836 from the lead wires 832 of the tubular member 806. For instance, a cutting device can be navigated alongside the tubular member 806, and the cutting device can be configured to cut a trench (e.g., a narrow channel) through any endothelization from the venous access site to the treatment site. The cutting device can be further configured to cut around the tubular member 806 and/or through the lead wires 832. In other embodiments, the electrodes 836 are fastened to the tubular member 806 using one or more engagement members (e.g., similar to the sutures 608), and the one or more engagement members can be cut by the cutting device. Other separation techniques are possible, including electrical decoupling, electrochemical decoupling, mechanical decoupling, etc.

Further, in some embodiments, the electrodes 836 are omitted, and the lead wires 832 may directly contact the expandable member 804 to at least partially transfer current to the expandable member 804. During decoupling of the expandable member 804 from the tubular member 806, a cutting device can be used to cut through the lead wires 832 to separate the lead wires 832 from the expandable member 804, e.g., as described above.

FIG. 9 illustrates a medical device assembly 902, in accordance with embodiments of the present technology. The medical device assembly 902 can be generally similar to any of the medical device assemblies discussed herein, except as described below. For instance, the medical device assembly 902 can include an expandable member 904 and a tubular member 906 configured to be releasably coupled with the expandable member 904 via one or more engagement members (not depicted). The coupling of the expandable member 904 and the tubular member 906 can be the same or similar to any of the coupling features and/or processes discussed above with respect to FIG. 2A–8.

In some embodiments, the medical device assembly 902 includes a substrate 938 (e.g., a flexible circuit board). The substrate 938 can be positioned within a lumen of the expandable member 904. For instance, the substrate 938 may be attached to an inner surface of the expandable member 904. In some embodiments, the substrate 938 includes a plurality of electrodes 910 configured to deliver therapeutic stimulation to surrounding tissue. The electrodes 910 can be generally similar to any of the electrodes described herein, such as the electrodes 210 of the medical device assembly 202 of FIG. 2. The substrate 938 can be configured to electrically couple the electrodes 910 to the tubular member 906 (or electrical components thereof). For instance, the substrate 938 can include a plurality of circuit traces 940, each circuit trace 940 coupled to a respective electrode 910. The circuit traces 940 may terminate at a trace joint 942 at a proximal end of the substrate 938. In some embodiments, the trace joint 942 is positioned within a conductor breakaway 944, and the conductor breakaway 944 is configured to receive the tubular member 906 to electrically couple the tubular member 906 (or electrical components thereof) to the electrodes 910. This may occur, for instance, when the tubular member 906 is coupled to the expandable member 904 via the one or more engagement members. The conductor breakaway 944 can take any suitable form configured to provide a severable electrical connection between internal conductors within the tubular member 906 and the electrodes 910 via the traces 940 and trace joint 942. For instance, a solder, weld, or adhesive joint can be provided that facilitates a mechanical and electrical connection that can be severed by rotation, retraction, cutting, or other disengagement of the tubular member 906 from the breakaway 944. In some implementations, the breakaway 944 can include thinned portions of conductors, perforations, grooves, notches, or other features configured to facilitate breakage at predefined locations. In various examples, the breakaway coupling illustrated here with respect to FIG. 9 can also be applied to other embodiments described herein, such as the medical device assemblies shown and described in FIG. 2A–7B. In such implementations, the breakaway coupling can be configured to detach such that the electrodes can remain with the implanted expandable member (e.g., stent) while the lead body is retracted.

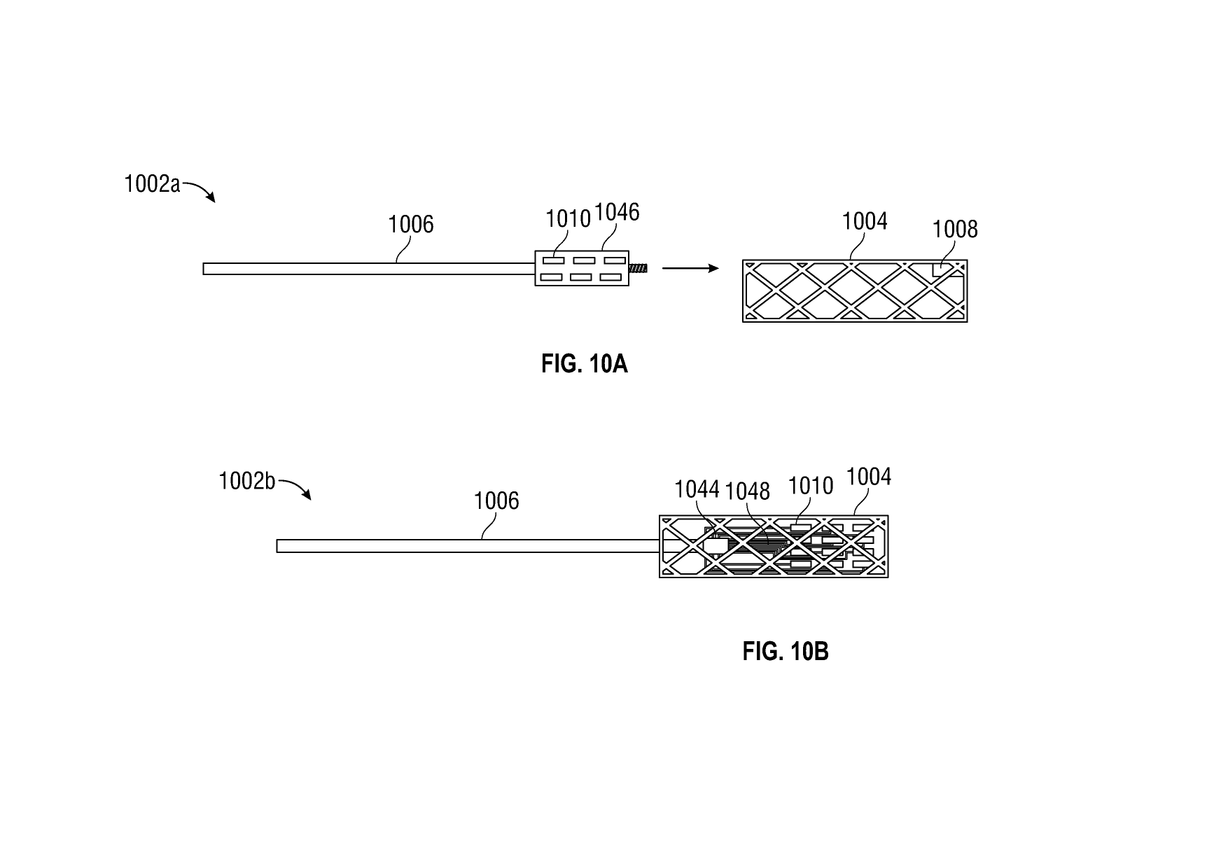

FIGS. 10A and 10B illustrate medical device assemblies 1002a and 1002b in accordance with embodiments of the present technology. The medical device assemblies 1002a and 1002b can be generally similar to any of the medical device assemblies discussed herein, except as described below. For instance, the medical device assemblies 1002a and 1002b can each include an expandable member 1004 and a tubular member 1006 configured to be releasably coupled with the expandable member 1004 via one or more engagement members 1008. The coupling of the expandable member 1004 and the tubular member 1006 can be the same or similar to any of the coupling features and/or processes discussed above with respect to FIG. 2A–9.

Referring first to FIG. 10A, in some embodiments, the medical device assembly 1002a further includes an electrode array 1046, such as an electrode paddle 1046. The electrode paddle 1046 can include a plurality of electrodes 1010 configured to deliver therapeutic stimulation to surrounding tissue. The electrodes 1010 can be generally similar to any of the electrodes discussed herein, such as the electrodes 210 of the medical device assembly 202 of FIG. 2. The electrode paddle 1046 can be positioned on an external surface of the tubular member 1006. For instance, the electrode paddle 1046 can be configured to wrap around the external surface of the tubular member 1006. In some embodiments, the electrode paddle 1046 is configured to self-expand. For instance, the electrode paddle 1046 may be delivered in a compressed configuration having a low-profile, and may unfurl at a treatment site, e.g., when the tubular member 1006 is coupled to the expandable member 1004.

The electrode paddle 1046 can be rectangular, circular, oblong, elongate, and/or any other suitable geometry for carrying the electrodes 1010. In some embodiments, the electrode paddle 1046 includes at least two, three, four, five, 6, 7, 8, 9, 10, 20, 50, or more electrodes 1010. Each of the electrodes 1010 carried by the electrode paddle 1046 can be coupled to a lead wire housed by the tubular member 1006. Accordingly, the electrodes 1010 may be selectively activated and deactivated, such that electrical stimulation can be varied as desired (e.g., patterned stimulation, etc.).

While FIG. 10A illustrates the electrode paddle 1046 as being positioned on the tubular member 1006, in some embodiments, the electrode paddle 1046 can be housed within a lumen of the expandable member 1004. Turning now to the medical device assembly 1002b of FIG. 10B, the electrode paddle 1046 is positioned at a distal end of the expandable member 1004. The electrode paddle 1046 can be in an expanded/deployed state, such that the electrode paddle 1046 is proximate an interior surface of the expandable member 1004. In some embodiments, the electrodes 1010 of the electrode paddle 1046 are electrically coupled to a plurality of lead wires 1048. The lead wires 1048 may be detachable and/or terminated at any point along the lead wires 1048 or at a conductor breakaway 1044 at or near a proximal end region of the expandable member 1004. In some embodiments, the conductor breakaway 1044 is configured to receive the tubular member 1006 to electrically couple the tubular member 1006 (or electrical components thereof) to the electrodes 1010. This may occur, for instance, when the tubular member 1006 is coupled to the expandable member 1004 via the one or more engagement members 1008.

### III.Detachment Methods

The treatment devices and systems provided herein can be used in treating a variety of medical conditions. Examples of methods suitable for use with any of the treatment devices and/or medical device assemblies described herein, such as those described above with respect to FIG. 2A–10B, will now be described. However, the methods described herein may additionally or alternatively be performed with any suitable variation of treatment device and/or medical device assembly in accordance with the present technology.

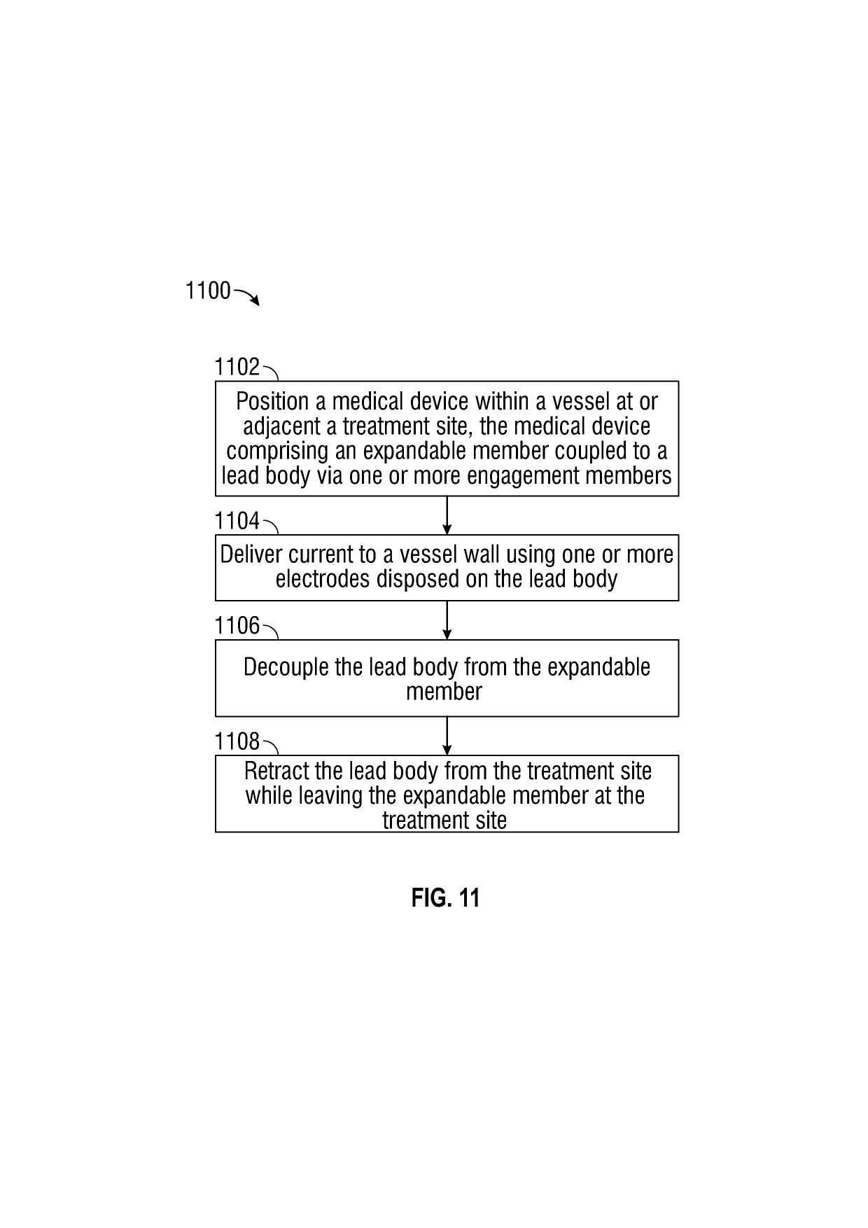

FIG. 11 is a flow chart of an example method 1100 for delivering therapeutic stimulation to a patient. In some embodiments, the method 1100 includes positioning a medical device within a vessel at or adjacent a treatment site 1102. The medical device assembly can be generally similar to any of the medical devices described herein, such as the medical device assembly 202 of FIG. 2. For instance, the medical device can include an expandable member, a lead body, and one or more engagement members. In some embodiments, the expandable member is configured to support, expand, and/or anchor to one or more vessel walls. The lead body can include a plurality of electrodes configured to stimulate surrounding tissue. The expandable member and the lead body may be releasably coupled to one another via the one or more engagement members. For instance, the one or more engagement members may include a receptacle positioned on the expandable member, the receptacle configured to receive the lead body, e.g., a distal tip of the lead body.

The method 1100 can further include delivering current to a vessel wall using the one or more electrodes of the lead body 1104. In some embodiments, the lead body comprises a plurality of lead wires, each lead wire coupled to a signal generator on a proximal side of the lead wire and a respective one of the one or more electrodes on a distal side of the lead wire. The electrodes may be in contact with the vessel wall. For instance, the electrodes may protrude from within a lumen of the expandable member toward the vessel wall. In some implementations, the electrodes are configured to stimulate a neural target, such as vagal nerve, pudendal nerve, or sciatic nerve. Alternatively, delivering current to the vessel wall may include delivering current to the expandable member. For instance, the lead wire may be configured to deliver current directly to the expandable member, and the expandable member may transfer the current to the vessel wall by contacting the vessel wall.

The method 1100 can further include decoupling the lead body from the expandable member 1106. Decoupling the lead body from the expandable member may include causing the one or more engagement members to transition from an engaged configuration to a disengaged configuration. For instance, in embodiments where the lead body is fastened into the one or more engagement members, the lead body may be rotated (e.g., twisted) to release the lead body from the expandable member. Additionally or alternatively, in embodiments where the lead body and the engagement members are locked together, such as with a pin, a stylet may be introduced into the lead body to displace the pin, thereby releasing the lead body from the expandable member. Further, in embodiments where the lead body and the expandable member are sutured together, proximal and/or distal displacement of the lead body relative to the expandable member may cause the sutures to break apart, releasing the lead body from the expandable member.

The method 1100 can further include retracting the lead body from the treatment site while leaving the expandable member at the treatment site 1108. The retraction can be a partial retraction or a full retraction. For instance, in some embodiments, the lead body is fully withdrawn from the vasculature. In some implementations, as described above with respect to FIG. 9, the electrodes may be detached from the lead body and remain with the expandable member at the treatment site 1108 after retraction of the lead body. Further, as described above with respect to FIG. 8, the electrodes may be detached from the lead body using a separation technique, such as by cutting around the lead body and through lead wires using a cutting device.

Optionally, the method 1100 can further include re-introducing the lead body to the treatment site. In some embodiments, re-introducing the lead body to the treatment site comprises distally advancing the lead body through the vasculature until the lead body abuts the one or more engagement members of the expandable member. Thereafter, the one or more engagement members may be configured to releasably couple the lead body to the expandable member using any of the engagement mechanisms and/or methods discussed herein.

### Conclusion

Although many of the embodiments are described above with respect to systems, devices, and methods for delivering electrical stimulation to neural targets, the technology is applicable to other applications and/or other approaches, such as delivering electrical stimulation elsewhere in the body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIG. 1A–11.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term "comprising" is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The present technology is illustrated, for example, according to various aspects described below. Various examples of aspects of the present technology are described as numbered Examples (1, 2, 3, etc.) for convenience. These are provided as examples and do not limit the present technology. It is noted that any of the dependent Examples may be combined in any combination, and placed into a respective independent Example. The other Examples can be presented in a similar manner.

Example 1. A medical device assembly comprising: an expandable member configured to be positioned in a blood vessel; a lead body comprising a plurality of electrodes in a distal end region of the lead body, wherein the electrodes are configured to deliver therapeutic stimulation to surrounding tissue; and one or more engagement members configured to releasably couple the lead body to the expandable member.

Example 2. The medical device assembly of any one of the preceding Examples, wherein the one or more engagement members comprise a receptacle coupled to the expandable member, the receptacle configured to receive at least a portion of the lead body.

Example 3. The medical device assembly of any one of the preceding Examples, wherein the lead body further comprises a distal tip, and wherein the receptacle is configured to receive the distal tip.

Example 4. The medical device assembly of any one of the preceding Examples, wherein the distal tip comprises a threaded tip.

Example 5. The medical device assembly of any one of the preceding Examples, wherein the distal tip is configured to be inserted in or released from the receptacle by twisting the lead body relative to the receptacle.

Example 6. The medical device assembly of any one of the preceding Examples, wherein the distal tip is insulated from the rest of the lead body.

Example 7. The medical device assembly of any one of the preceding Examples, wherein the lead body further comprises a slot, and wherein the receptacle comprises a spring-loaded pin configured to engage the slot.

Example 8. The medical device assembly of any one of the preceding Examples, wherein the spring-loaded pin is configured to be released from the slot using a stylet inserted through the lead body.

Example 9. The medical device assembly of any one of the preceding Examples, wherein the one or more engagement members further comprise a second receptacle, and wherein the second receptacle comprises a second spring-loaded pin configured to engage a second slot of the lead body.

Example 10. The medical device assembly of any one of the preceding Examples, wherein the receptacle is on a radially outer side of the expandable member.

Example 11. The medical device assembly of any one of the preceding Examples, wherein the receptacle is positioned in a lumen of the expandable member.

Example 12. The medical device assembly of any one of the preceding Examples, wherein the receptacle is positioned at a distal portion of the expandable member.

Example 13. The medical device assembly of any one of the preceding Examples, wherein the one or more engagement members comprise one or more bioabsorbable sutures.

Example 14. The medical device assembly of any one of the preceding Examples, further comprising an attachment coupled to the lead body, wherein the bioabsorbable sutures are threaded through the attachment.

Example 15. The medical device assembly of any one of the preceding Examples, wherein the attachment comprises one or more of tines, barbs, hooks, or rods for securing the lead body to the expandable member.

Example 16. The medical device assembly of any one of the preceding Examples, wherein the bioabsorbable sutures are directly coupled to both the expandable member and the lead body.

Example 17. The medical device assembly of any one of the preceding Examples, wherein the bioabsorbable sutures are configured to release the lead body upon retraction of the lead body relative to the expandable member.

Example 18. The medical device assembly of any one of the preceding Examples, wherein the expandable member comprises a plurality of struts, and wherein the electrodes are positioned in between the struts.

Example 19. The medical device assembly of any one of the preceding Examples wherein the lead body is isodiametric.

Example 20. The medical device assembly of any one of the preceding Examples, wherein the lead body comprises platinum.

Example 21. A medical device assembly comprising: an expandable member configured to be positioned in a blood vessel; a plurality of electrodes coupled to the expandable member; a tubular member comprising a plurality of wires configured to releasably engage the electrodes; and one or more engagement members configured to releasably couple the tubular member to the expandable member.

Example 22. The medical device assembly of any one of the preceding Examples, wherein the one or more engagement members comprise a receptacle coupled to the expandable member, the receptacle configured to receive at least a portion of the tubular member.

Example 23. The medical device assembly of any one of the preceding Examples, wherein the tubular member further comprises a distal tip, and wherein the receptacle is configured to receive the distal tip.

Example 24. The medical device assembly of any one of the preceding Examples, wherein the distal tip comprises a threaded tip.

Example 25. The medical device assembly of any one of the preceding Examples, wherein the distal tip is configured to be inserted in or released from the receptacle by twisting the tubular member relative to the receptacle.

Example 26. The medical device assembly of any one of the preceding Examples, wherein the distal tip is insulated from the rest of the tubular member.

Example 27. The medical device assembly of any one of the preceding Examples, wherein the tubular member further comprises a slot, and wherein the receptacle comprises a spring-loaded pin configured to engage the slot.

Example 28. The medical device assembly of any one of the preceding Examples, wherein the spring-loaded pin is configured to be released from the slot using a stylet inserted through the tubular member.

Example 29. The medical device assembly of any one of the preceding Examples, wherein the one or more engagement members further comprise a second receptacle, and wherein the second receptacle comprises a second spring-loaded pin configured to engage a second slot of the tubular member.

Example 30. The medical device assembly of any one of the preceding Examples, wherein the receptacle is on a radially outer side of the expandable member.

Example 31. The medical device assembly of any one of the preceding Examples, wherein the receptacle is positioned in a lumen of the expandable member.

Example 32. The medical device assembly of any one of the preceding Examples, wherein the receptacle is positioned at a distal portion of the expandable member.

Example 33. The medical device assembly of any one of the preceding Examples, wherein the one or more engagement members comprise one or more bioabsorbable sutures.

Example 34. The medical device assembly of any one of the preceding Examples, further comprising an attachment coupled to the tubular member, wherein the bioabsorbable sutures are threaded through the attachment.

Example 35. The medical device assembly of any one of the preceding Examples, wherein the attachment comprises one or more of tines, barbs, hooks, or rods for securing the tubular member to the expandable member.

Example 36. The medical device assembly of any one of the preceding Examples, wherein the bioabsorbable sutures are directly coupled to both the expandable member and the tubular member.

Example 37. The medical device assembly of any one of the preceding Examples, wherein the bioabsorbable sutures are configured to release the tubular member upon retraction of the tubular member relative to the expandable member.

Example 38. The medical device assembly of any one of the preceding Examples, wherein the expandable member comprises a plurality of struts, and wherein the electrodes are positioned in between the struts.

Example 39. The medical device assembly of any one of the preceding Examples, wherein the tubular member is isodiametric.

Example 40. The medical device assembly of any one of the preceding Examples, wherein the tubular member comprises platinum.

Example 41. A method comprising: disposing a medical device within a vessel at or adjacent to a treatment site, the medical device comprising: an expandable member, a lead body comprising a plurality of electrodes in a distal end region of the lead body, and one or more engagement members releasably coupling the lead body to the expandable member; and decoupling the lead body from the expandable member via the one or more engagement members.

Example 42. The method of any one of the preceding Examples, further comprising delivering electrical current to a vessel wall using the electrodes.

Example 43. The method of any one of the preceding Examples, further comprising, after the decoupling, withdrawing the lead body from the vessel while leaving the expandable member at or adjacent to the treatment site.

Example 44. The method of any one of the preceding Examples, further comprising after withdrawing the lead body from the treatment site, reintroducing the lead body to the treatment site and recoupling the lead body to the expandable member at the treatment site.

Example 45. The method of any one of the preceding Examples, further comprising separating the plurality of electrodes from the lead body.

Example 46. The method of Example 45, wherein the separation includes cutting around the lead body to separate the plurality of electrodes from the leady body.

1. A medical device assembly comprising:
an expandable member configured to be positioned in a blood vessel;
a lead body comprising a plurality of electrodes in a distal end region of the lead body, wherein the electrodes are configured to deliver therapeutic stimulation to surrounding tissue; and
one or more engagement members configured to releasably couple the lead body to the expandable member.

2. The medical device assembly of claim 1, wherein the one or more engagement members comprise a receptacle coupled to the expandable member, the receptacle configured to receive at least a portion of the lead body.

3. The medical device assembly of claim 2, wherein the lead body further comprises a distal tip, and wherein the receptacle is configured to receive the distal tip.

4. The medical device assembly of claim 3, wherein the distal tip comprises a threaded tip.

5. The medical device assembly of claim 4, wherein the distal tip is configured to be inserted in or released from the receptacle by twisting the lead body relative to the receptacle.

6. The medical device assembly of claim 2, wherein the lead body further comprises a slot, and wherein the receptacle comprises a spring-loaded pin configured to engage the slot.

7. The medical device assembly of claim 2, wherein the receptacle is positioned in a lumen of the expandable member.

8. The medical device assembly of claim 1, wherein the one or more engagement members comprise one or more bioabsorbable sutures.

9. A medical device assembly comprising:
an expandable member configured to be positioned in a blood vessel;
a plurality of electrodes coupled to the expandable member;
a tubular member comprising a plurality of wires configured to releasably engage the electrodes; and
one or more engagement members configured to releasably couple the tubular member to the expandable member.

10. The medical device assembly of claim 9, wherein the one or more engagement members comprise a receptacle coupled to the expandable member, the receptacle configured to receive at least a portion of the tubular member.

11. The medical device assembly of claim 10, wherein the tubular member further comprises a distal tip, and wherein the receptacle is configured to receive the distal tip.

12. The medical device assembly of claim 11, wherein the distal tip comprises a threaded tip.

13. The medical device assembly of claim 12, wherein the distal tip is configured to be inserted in or released from the receptacle by twisting the tubular member relative to the receptacle.

14. The medical device assembly of claim 10, wherein the tubular member further comprises a slot, and wherein the receptacle comprises a spring-loaded pin configured to engage the slot.

15. The medical device assembly of claim 10, wherein the receptacle is positioned in a lumen of the expandable member.

16. The medical device assembly of claim 9, wherein the one or more engagement members comprise one or more bioabsorbable sutures.

17. A method comprising:
disposing a medical device within a vessel at or adjacent to a treatment site, the medical device comprising:
an expandable member,
a lead body comprising a plurality of electrodes in a distal end region of the lead body, and
one or more engagement members releasably coupling the lead body to the expandable member; and
decoupling the lead body from the expandable member via the one or more engagement members.

18. The method of claim 17, further comprising delivering electrical current to a vessel wall using the electrodes.

19. The method of claim 17, further comprising, after the decoupling, withdrawing the lead body from the vessel while leaving the expandable member at or adjacent to the treatment site.

20. The method of claim 19, further comprising after withdrawing the lead body from the treatment site, reintroducing the lead body to the treatment site and recoupling the lead body to the expandable member at the treatment site.
